(19) United States
(12) Patent Application Publication
(10) Pub. No.: US 2020/0370021 A1
(43) Pub. Date: Nov. 26, 2020

(54) SYNTHETIC TUMOR MICROENVIRONMENT TO REGULATE CANCER CELL BEHAVIOR

(71) Applicants: CURE BIO Co., Ltd., Seoul (KR); Kollodis BioSciences Inc., North Augusta, SC (US); AMOLIFESCIENCE CO., LTD., Seoul (KR)

(72) Inventors: Myung-Yeol Lee, Seoul (KR); Chi-Uk In, Seoul (KR); Sang-Jae Lee, North Augusta, SC (US); Bong-Jin Hong, North Augusta, SC (US); Min-Chul Park, Seoul (KR)

(21) Appl. No.: 16/900,830

(22) Filed: Jun. 12, 2020

Related U.S. Application Data
(63) Continuation-in-part of application No. 16/546,966, filed on Aug. 21, 2019.
(60) Provisional application No. 62/720,800, filed on Aug. 21, 2018.

(51) Int. Cl. C12N 5/09

(52) U.S. Cl. CPC C12N 5/0693; C12N 2539/10; C12N 2535/00; C12N 2533/50

(57) ABSTRACT

Described are methods and devices for enrichment and in situ expansion of circulating tumor cells (CTCs) from biological samples. The methods may include detecting at least one or more of cell adhesion molecules as epithelial mesenchymal transition (EMT) biomarker. Also described is device for detecting or enriching CTCs. The surface of the device may provide at least one or more of cell binding ligands such as ECM or cadherin derived peptide motif to detect the EMT biomarker. Also described is a surface to remove leukocytes from biological samples, leading to efficient enrichment of CTCs from the biological samples.

### CROSS-REFERENCE TO RELATED APPLICATIONS

This application is a continuation-in-part of pending U.S. patent application Ser. No. 16/546,966, filed Aug. 21, 2019, which claims the benefit of U.S. Provisional Patent Application Serial No. 62/720,800, filed Aug. 21, 2018, the disclosure of each of which is hereby incorporated herein in its entirety by this reference.

### TECHNICAL FIELD

The application relates to synthetic tumor microenvironment(s) for enriching and culturing tumor cells including cancer stem cells and metastatic tumor cells. More particularly, the disclosure provides tumor microenvironment surface and method for enriching and in situ culturing circulating tumor cells on a fully defined tumor microenvironment surface.

### BACKGROUND

Metastasis is the major cause for cancer-related death, and is a multi-step process which includes local tumor cell invasion, entry into the vasculature (intravasation) and survival in the circulatory system, followed by the exit of carcinoma cells from the circulation (extravasation) and colonization at the distal sites (see, A. Eger, W. Mikulits, Models of epithelial-mesenchymal transition, Drug Discov. Today: Dis. Models. 2005; 2:57-63).

Circulating tumor cells (CTCs) represent an intermediate stage of metastasis. CTCs derived from actively invading cells acquire key properties required for metastatic spread. Therefore, the metastatic tumor cell must acquire the ability to emigrate from the primary tumor—meaning it must shed cell-cell and cell-matrix interactions, become motile, and acquire plasticity in order to mechanically navigate the tumor stroma. It must then acquire invasive capabilities, allowing it to degrade the surrounding extracellular matrix and allow escape from the primary tumor mass (see, Lindsay J Talbot, et al., epithelial-mesenchymal transition, the tumor microenvironment, and metastatic behavior of epithelial malignancies, Int. J. Biochem. Mol. Biol. 2012; 3(2):117-136).

In clinical practice, CTC enumeration has been recognized for its prognostic value, and technologies for the enrichment and isolation of CTCs have ranged from the simple to the sophisticated.

The immunomagnetic techniques, most widely used for CTC detection, are based on labeling CTCs using antigen-specific antibodies linked to magnetic beads. Currently, clinically acceptable markers for detection of CTC are EpCAM and CK (Cytokerain), but these markers can detect only epithelial CTC, but not epithelial-mesenchymal transformed (EMT) CTC and other non-epithelial CTC that originate from mesenchymal tumors, which constitute about 10% of adult and about 20% of pediatric cancer types.

Microfiltration method is based on trapping the major epithelial cells (on average 25 μm) while passing small leukocytes (on average 15 μm) through the pores of defined size and shape. However, the overlap of size in leukocyte and CTC limits its application.

Therefore, there is a need to develop novel approach to overcome the drawbacks of the immunomagnetic and microfiltration method described above.

### BRIEF SUMMARY

Described are multiple biomarkers, including cell binding molecules such as extracellular matrix proteins, based CTC capturing, classification, and in situ expansion technology. The multiple biomarkers in the disclosure are main components of tumor microenvironment. Many of the steps in metastasis formation require specific interactions with the extracellular matrix, and the nature and degree of these matrix interactions will change from step to step during the metastatic process. Described are specific interactions as biomarkers between cell adhesion receptors and cell binding molecules in tumor cells undergoing changes in extracellular microenvironment from step to step during the metastatic process.

In one aspect, a synthetic tumor microenvironment surface that specifically binds to cancer cells of interest, particularly circulating tumor cells in the blood, is provided. The tumor microenvironment surface can bind specific cell adhesion receptors including integrins, cadherins, and EpCAM highly expressed in non-metastatic or metastatic tumor cells. For example, collagen IV could be a biomarker in less-metastatic cancer cells while collagen type I could be a biomarker in metastatic cancer cells.

In another aspect, a device to isolate & classify from mixed cell population such as blood sample, and to expand the captured or isolated tumor cells is provided. After cells are arrayed in accordance with its corresponding specific tumor microenvironment, the arrayed cells may be characterized or maintained in culture for a period of time sufficient to determine the response to a stimulus such as drug efficacy.

In another aspect, a synthetic tumor microarray to identify cell adhesion molecules as potential biomarker to detect and/or isolate tumor cells at specific metastatic stage for diagnostic or prognostic application is provided.

In other aspects, there is provided a method of isolating, classifying, and in situ expanding circulating tumor cells from mixed cell population. Current CTC expansion efficiency is about or less 20% and its maintenance period of primary cancer cells is less than 6 months. The CTC expansion efficiency is about 100% in the disclosure, allowing drug efficacy test on circulating tumor cells derived from patient blood.

### BRIEF DESCRIPTION OF DRAWINGS

FIGS. 1A and 1B represent schematic cell capturing island formation process via EDC/S-NHS mediated protein particle formation, followed by its precipitation to form island like morphology as seen in FIG. 1A, and FIG. 1B shows the Cell Capturing Island as a synthetic tumor microenvironment surface where cell adhesion peptide specifically binding to tumor cell surface receptor as shown in the right side.

FIG. 2 represents the SEM image of Cell Capturing Island formed on a neutral substrate. On average 2 μm sized protein particles (white particles) presenting cell adhesion peptide were formed as Island on the neutral substrate. Average distance between protein particles was 20 μm.

FIGS. 3A and 3B represent the surface captured metastatic tumor cells when integrin α2β1 binding peptide motif was present on the Cell Capturing Island as seen in FIG. 3A (C) while no tumor cells were captured on both surfaces in FIG. 3A (A) and (B) due to lack of binding capacity. FIG. 3B shows the binding capacity of 6 different cell adhesion peptide motifs, E-cadherin derived peptide in (A), E-cadherin derived peptide in (B), E-cadherin derived peptide in (C), E-cadherin derived peptide in (D), E-cadherin derived peptide in (E), E-cadherin derived peptide in (F).

FIGS. 4A and 4B represent the layout of synthetic microenvironment surface array to screen an optimal tumor microenvironment. FIG. 4A illustrates the layout of epithelial surface to bind epithelial markers and FIG. 4B indicates mesenchymal surface to capture cells with mesenchymal properties. The surface of each well presents different integrin binding, E-cadherin binding, and EpCAM binding peptide motif.

FIGS. 5A-5D represent the adhesion profiling of non-metastatic cancer cells on the synthetic tumor microenvironment surface where different cell binding motifs are presented. Epithelial surface presenting cell binding peptide motif to epithelial markers enriched MCF-7, non-metastatic cell, and mesenchymal surface for mesenchymal markers enriched MDA-MB-231, a metastatic cancer cell.

FIGS. 6A and 6B represent the adhesion profiling of leukocytes on two synthetic tumor microenvironment surfaces, epithelial and mesenchymal surfaces. Leukocytes were poorly enriched compared to tumor cells. It is noted that syndecan binding motif (RKRLQVQLSIRT (SEQ ID NO:63)) and tenascin motif binding to integrin α9β1 captured leukocytes, but its binding capacity or efficient was low.

FIGS. 7A and 7B represent the layout of synthetic tumor microenvironment surface presenting combination of two different cell binding peptide motifs to bind to two different epithelial markers; cell adhesion receptor integrin αvβ6 and integrins (α1β1, α3β1, α6β1). The combinatorial presentation of two different cell binding motif did not show significant synergistic effect on tumor cell capturing as observed in FIG. 7B.

FIGS. 8A and 8B represent the layout of synthetic tumor microenvironment surface presenting combination of two different cell binding peptide motifs to bind to two different mesenchymal markers; cell adhesion receptor integrin α2β1 and integrin α6β1/E-cadherin. The combinatorial presentation of two different cell binding motif showed significant synergistic effect on tumor cell capturing as seen in FIG. 8B.

FIG. 9 represents the optical microscopic image of captured non-metastatic tumor cells, MCF-7, on the surface presenting integrin α1β1 binding motif GFPGER (SEQ ID NO:21) and E-cadherin binding motif DQNDN (SEQ ID NO:31) or EpCAM binding motif RGDPAYQGRFL (SEQ ID NO:34). The capturing efficiency was poor as expected because the mesenchymal integrin does not strongly support the adhesion of epithelial phenotypic cells.

FIG. 10 represents the optical microscopic image of captured metastatic tumor cells, MDA-MB-231, on the surface presenting integrin α1β1 binding motif GFPGER (SEQ ID NO:21) and E-cadherin binding motif DQNDN (SEQ ID NO:31) or EpCAM binding motif RGDPAYQGRFL (SEQ ID NO:34). Unlike MCF-7, the mesenchymal phenotypic cells strongly attached to the mesenchymal surface.

FIGS. 11A and 11B represent the layout of array where different leukocyte integrin binding peptide motifs are present in each well (FIG. 11A), and the effect of cell binding ligand on leukocytes capturing (FIG. 11B).

FIGS. 12A and 12B represent the effect of cell binding ligand on the expression level of cancer stemness marker in non-metastatic (MCF-7) and metastatic cancer cell (MDA-MB-231) when in situ expanded on the tumor microenvironment surface that captured cancer cells.

### DETAILED DESCRIPTION

The disclosure is directed to a synthetic tumor microenvironment composition, surface, device and kits for cancer cell-specific capturing or sorting of captured tumor cells from a mixed cell population. Aspect of the disclosure combine a substrate with at least one or more tumor microenvironment-forming molecules in order to mimic in vivo tumor microenvironment.

As used herein “tumor microenvironment” refers to physical and/or biochemical cues, surrounding tumor cells in an organism or in the laboratory. Extracellular matrix proteins, growth factors, cytokines and parameters such as pH, ionic strength and gas composition, and the like surrounding tumor cells are the biochemical cues.

As used herein “synthetic tumor microenvironment” refers to an engineered surface of a substrate to present the biochemical cues to mimic in vivo tumor microenvironment as a key component of tumor microenvironment. The molecules for biochemical cues may be immobilized to a substrate. The biochemical cues in the disclosure include cell binding ligand such as extracellular matrix (ECM), cadherin, or any peptide motif to bind to cells via cell adhesion molecules such as integrin, cadherin, or EpCAM. The cell binding ligand may be naturally occurring or recombinant, or its mimetic such as core peptide motif derived from ECM or cadherin.

The extracellular matrix (ECM) is a collection of extracellular molecules secreted by support cells that provides structural and biochemical support to the surrounding cells. Many cells bind to components of the extracellular matrix. Cell adhesion can occur in two ways; by focal adhesions, connecting the ECM to actin filaments of the cell, and hemidesmosomes, connecting the ECM to intermediate filaments such as keratin. This cell-to-ECM adhesion is regulated by cell adhesion molecule known as integrins.

Cell adhesion molecules (CAMs) are proteins located on the cell surface involved in binding with other cells or with the extracellular matrix (ECM) in the process called cell adhesion. Main cell adhesion molecules are integrins, selectins, adhesion molecules belonging to the immunoglobulin superfamily, cadherins, and the CD44 family. All cell adhesion molecules bind to other cells or matrix components through their interaction with appropriate counter- structures, referred to as ligands. (See, Paul Murray, et al., Cell adhesion molecules, BMJ 1999; 319:332.) In the disclosure, cell adhesion molecules refer to integrins, immunoglobulin superfamily, cadherins, and epithelial cell adhesion molecule.

Epithelial cell adhesion molecule (“EpCAM”) is a transmembrane glycoprotein primarily known to mediate homotypic cell contacts in epithelia tissues. Because EpCAM expression is limited to normal and malignant epithelia, it has been used as diagnostic marker for the detection of carcinoma cells in mesenchymal organs such as blood, bone marrow or lymph nodes. (See, Laura Keller, et al., Biology and clinical relevance of EpCAM, Cell Stress, Vol. 3, No. 6, pp. 165-180.)

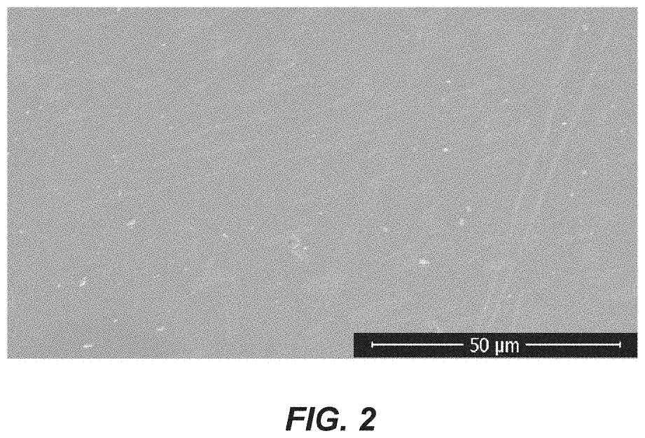

A surface for synthetic tumor microenvironment may be selected from any electrically neutral or hydrophobic surface, forcing cells into a suspended state. When particles' surface presenting cell binding ligand, as illustrated in FIG. 2, is coated on the neutral or hydrophobic surface, cells expressing cell adhesion molecules to bind the cell binding ligand can be captured or sorted while other cells are forced into a suspended state.

Any hydrophobic or neutral substrate, including but not limited to, synthetic or natural polymer such as protein, glass, metal, or its hybrid can be used for the synthetic tumor microenvironment surface in the disclosure. In one embodiment, any hydrophobic substrate such as polystyrene (PS), silicone, Teflon, or polyvinyliden fluoride (PVDF) substrate is used to create a synthetic tumor microenvironment. In another embodiment, as a neutral surface, examples of commercially available substrate include Ultra-Low Attachment Surface marketed by Corning, Inc. (NY, USA).

As used herein “Cell Capturing Island” refers to particles presenting cell binding ligand specifically binding to cell adhesion molecules highly expressed in tumor cells are coated on neutral or hydrophobic surface to form island-like morphology wherein the tumor cells are captured or sorted by the cell binding ligand presented on the island surface.

A composition to make Cell Capturing Island is generally composed of two components, one is a substrate protein to form a particle and the other is a cell binding component presented on the particle's surface.

Any suitable substrate protein including but not limited to fibrin, elastin, mussel adhesive protein may be used as the substrate protein to present cell binding component. Preferably, the substrate protein is a recombinant mussel adhesive protein.

Any suitable recombinant mussel adhesive protein may be used as the substrate protein in the disclosure. Examples of commercially available substrate proteins include MAPTrix™ ECM marketed by Kollodis BioSciences, Inc. (North Augusta, S.C.). An optional third component is a biocompatible polymer (e.g., polyethylene glycol or polyvinylalcohol), which may be added to the compositions to enhance their physicomechanical characteristics such as physical or mechanical properties of a customizable tumor microenvironment.

The MAPTrix™, developed by Kollodis BioSciences Inc. (North Augusta, S.C.), are predesigned mussel adhesive protein or barnacle-based extracellular component mimetics. The mussel adhesive proteins were recombinantly functionalized with a variety of ECMs-, cadherins-, or other ligands derived peptides in order to mimic the bioactivity of naturally occurring cell binding ligand such as ECMs, cadherins, or soluble factors such as cytokines such as IL-6 which were demonstrated to have a similar bioactivity to natural or recombinant ECMs, cadherins, or soluble factors in primary cell cultures as compared to various natural or recombinant ECM, cadherin or cytokine proteins. The pre-designed MAPTrix™ mimetics are highly advantageous for creating synthetic tumor microenvironment. For example, it provides for the design of cancer cell-specific or user-defined regulation of extracellular microenvironments to emulate the native tumor microenvironment in terms of biochemical cues.

The MAPTrix™ is a fusion protein comprising a first peptide of mussel foot protein FP-5 that is selected from the group consisting SEQ ID NOS:1-4, or barnacle-derived adhesive protein and a second peptide of at least one selected from the group consisting of mussel FP-1 selected from the group consisting of SEQ ID NOS:6-8, mussel FP-2 (SEQ ID NO:9), mussel FP-3 selected from the group consisting of SEQ ID NOS:10, 11, mussel FP-4 (SEQ ID NO:12), mussel FP-6 (SEQ ID NO:13) and fragment thereof, and the second peptide is linked to C-terminus, N-terminus or C- and N-terminus of the FP-5. Preferably, the second peptide is the FP-1 comprising an amino acid sequence of SEQ ID NO:8, and its fusion protein has SEQ ID NO:14 or SEQ ID NO:15.

Any cell binding ligand to bind to specific cell adhesion molecules highly expressed in tumor cells can be a cell binding component to form Cell Capturing Island. Cell binding ligand can be selected from integrin binding, cadherin binding, or EpCAM binding molecules. The cell binding ligand such as ECM protein, cadherin, or EpCAM can be a natural or recombinant cell binding molecule or its derived domain including core motif that binds to specific cell adhesion molecule such as integrin, or its mimetic, can be used in the disclosure. The cell binding ligand component for Cell Capturing Island comprises of the substrate protein, recombinantly or chemically, functionalized with at least one or more peptide motifs derived from a variety of the cell binding ligand.

Generally, Cell Capturing Island include extracellular matrix mimetic, cadherin mimetic, EpCAM binding peptide, or its combination which are recombinantly or chemically incorporated into a substrate protein.

In the disclosure, cell binding ligand components including integrin binding motif, cadherin binding motif, or EpCAM binding peptide motif are recombinantly incorporated into the fusion protein of mussel adhesive protein to further enhance the beneficial effect of the tumor environment mimic on capturing or sorting of tumor cells of interest.

Provided is a surface coated with Cell Capturing Island that present a synthetic tumor microenvironment. Un-coated surface would force any cells to be suspended while only cells binding to the synthetic tumor microenvironment are captured. The synthetic tumor microenvironment surface in the disclosure provides at least one or more cell binding component to capture or sort tumor cells. In one embodiment, a synthetic tumor microenvironment surface presents integrin binding peptide motif to capture tumor cell highly expressing integrin which is specifically binding the integrin binding peptide motif. In another embodiment, a synthetic tumor microenvironment surface presents two different integrin binding peptide motifs to capture tumor cells highly expressing two different integrins binding specifically to the integrin binding peptides, respectively. In another embodiment, a synthetic tumor microenvironment surface presents two different cell binding components to capture tumor cells highly expressing two different cell adhesion molecules, for example integrin and cadherin, integrin and EpCAM, or cadherin and EpCAM.

Metastasis is a complex process during which cancer cells migrate away from the primary tumor, gain access to the circulation, and subsequently home to distant organs. Recent studies indicate that the metastatic process is not entirely linked to tumor growth, per se, but is controlled by other factors and can occur from early lesion (Y. Husemann, et al., (2008) Systemic spread is an early step in breast cancer, Cancer Cell 13, 58-68; C. K. Mikael, et al., Epithelial-mesenchymal transition in cancer metastasis through the lymphatic system, Mol. Oncol. 2017, 11(7) 781-791). The molecular pathways regulating invasion and motility as well as the specific mode of invasion employed by a cancer cell is largely cell-type specific in addition to the influence of the microenvironment. (Integrins in Tumorigenesis and Cancer Cell Invasion-Thesis.) Within the tumor microenvironment, changes in cancer cell-extracellular matrix (ECM) interactions influence each stage of the metastatic cascade, from the loss of basement membrane adhesion to colonization of distant sites.

The invasive front of the tumor in early stage of metastasis exhibits an epithelial-mesenchymal transition (EMT) phenotype associated with a loss of epithelial markers and cell-cell junctions and increased expression of mesenchymal markers (see, R. Y. Huang, et al, 2012, Early events in cell adhesion and polarity during epithelial-mesenchymal transition, J. Cell Sci. 125:4417-22 31; T. Brabletz, et al. 2001, Variable beta-catenin expression in colorectal cancers indicates tumor progression driven by the tumor environment, PNAS 98:10356-61). Several integrins are upregulated by TGF-β1 during the EMT process. Epithelial markers such as E-cadherin, laminin α1 were downregulated while mesenchymal markers such as N-cadherin, fibronectin, vitronectin were activated (see, A. M. Fahymy, et al, aV integrins and TGF-β induced EMT: a circle of regulation, J. Cell. Mol. Med. 2012, 16(3):445-55; S. Mori, et al., Enhanced Expression of Integrin αvβ3 Induced by TGF-β Is Required for the Enhancing Effect of Fibroblast Growth Factor 1 (FGF1) in TGF-β-Induced Epithelial-Mesenchymal Transition (EMT) in Mammary Epithelial Cells, PLoS ONE 10(9):e0137486).

Integrin αvβ6 is a transmembrane receptor composed of non-covalently linked αv and β6 subunits, where the β6 subunit partners exclusively with αv and is expressed only in epithelial tissues (see, R. O. Hynes RO (2002) Integrins: bidirectional, allosteric signaling machines, Cell 110:673-687). αvβ6 is concentrated in poorly differentiated tumors proximal to invading cancer margins, and has been identified as a prognostic indicator of poor survival in CRC (R. C. Bates, et al. (2005), Transcriptional activation of integrin beta6 during the epithelial- mesenchymal transition defines a novel prognostic indicator of aggressive colon carcinoma, J. Clin. Invest. 115:339-347), gastric adenocarcinoma (see, Z. Zhuang, et al. (2013), Clinical significance of integrin αvβ6 expression effects on gastric carcinoma invasiveness and progression via cancer-associated fibroblasts, Med. Oncol. 30:013-0580), where the CRC and gastric carcinoma studies were based on tumor Stage I through IV and the cervical squamous carcinoma was on patients identified as FIGO Stage IA through IIB. Overall, the expression of αvβ6 is suggested to be involved in the earlier stages of tumor progression rather than the later stages (S. B. Ahn, et al. (2014), Correlations between Integrin αvβ6 Expression and Clinico-Pathological Features in Stage B and Stage C Rectal Cancer, PLoS ONE 9(5):e97248).

The disclosure provides a synthetic tumor microenvironment surface that presents cell binding motif to adhere integrin αvβ6 highly expressed in early stage of metastasis. Any cell binding motif for integrin αvβ6 can be used in the disclosure. Generally, RGD (SEQ ID NO:15) containing motif is preferred. For example, RGD-SGSGSG-RGD-SGSGSG-RGD (SEQ ID NO:16) motif can be used to capture or sort tumor cells highly expressing integrin αvβ6. In one embodiment, a synthetic tumor microenvironment surface presenting RGD-SGSGSG-RGD-SGSGSG-RGD (SEQ ID NO:16) or MNYYSNS (SEQ ID NO:17) is provided to capture early stage metastatic tumor cells.

Changes in ECM composition, such as the increased expression of fibronectin, vitronectin and type I collagen seen during EMT, can switch integrins from an inactive low affinity to an active high affinity ligand binding state (see, Y. Imamich, et al., Signaling pathways involved in collagen-induced disruption of the E-cadherin complex during epithelial-mesenchymal transition, Cells Tissues Organs. 2007; 185:180-90). Type I collagenous activity, which is very rare in the benign tumors of the breast, large intestine, and stomach is at an abundant amount in the malignant tumors of the same organs (see, K. Kessenbrock et al., Matrix metalloproteinases: regulators of the tumor microenvironment, Cell. 2010; 141:52-67; Serdar Altinay, Is Extracellular Matrix a Castle Against to Invasion of Cancer Cells, Intech 2016, 23-42).

The disclosure provides a synthetic tumor microenvironment surface that presents peptide motifs derived from N-cadherin, fibronectin, vitronectin, or its combination to selectively isolate tumor cells undergoing EMT process from a mixed cell population. In one embodiment, the synthetic tumor microenvironment surface presents fibronectin-derived peptide motif, vitronectin-derived peptide motif, or type I collagen-derived peptide motif in order to enrich tumor cells undergoing EMT process from other cells. Type I collagen generally binds to integrin α2β1. In the disclosure, a synthetic tumor microenvironment surface presents cell binding motif to selectively bind to integrin α2β1.

Any suitable α2β1 integrin binding motif can be selected from GLSGER (SEQ ID NO:18), GASGER (SEQ ID NO:19), GQRGER (SEQ ID NO:20), GFPGER (SEQ ID NO:21), GLPGER (SEQ ID NO:22), DGEA (SEQ ID NO:23), GPAGKDGEAGAQG (SEQ ID NO:24), TAGSCLRKFSTM (SEQ ID NO:25), MFKKPTPSTLKAGELR (SEQ ID NO:26), LAGSCLARFSTM (SEQ ID NO:27), GEFYFDLRLKGDK (SEQ ID NO:28), or its combination of two or more α2β1 integrin binding motifs. In one embodiment, α2β1 integrin binding motif is GFPGER (SEQ ID NO:21) to selectively capture metastatic tumor cells from non-metastatic tumor cells.

Also described is a synthetic tumor microenvironment surface that presents an epithelial marker such as E-cadherin or EpCAM binding motif to enrich epithelial like tumor cells from mixed cell population. Any suitable E-cadherin binding motif can be selected from SHAVSS (SEQ ID NO:29), LFSHAVSSNG (SEQ ID NO:30), DQNDN (SEQ ID NO:31), ADTPPV (SEQ ID NO:32), QGADTPPVGV (SEQ ID NO:33), LRAHAVDVNG (SEQ ID NO:64) or its combination of two or more E-cadherin binding motifs. Any suitable EpCAM binding molecule is selected from RGDPAYQGRFL (SEQ ID NO:34), YEVHTYYLD (SEQ ID NO:35), or its combination. In one embodiment, E-cadherin binding motif DQNDN (SEQ ID NO:31) is presented to capture non-metastatic tumor cells from mixed cell population. In another embodiment, EpCAM binding motif RGDPAYQGRFL (SEQ ID NO:34) is presented to capture non-metastatic tumor cells from mixed cell population.

Also described is a synthetic tumor microenvironment surface that presents multiple, simultaneously or selectively binding event of multiple ligands to multiple receptors in tumor cells. In some aspect of the disclosure, the multiple binding effect is accomplished by presenting peptide motifs on the substrate of the cell capturing or sorting surface. In one embodiment, α2β1 integrin binding motif and E-cadherin binding motifs are simultaneously presented to bind multiple receptors in tumor cells.

In another embodiment, α2β1 integrin binding motif GFPGER (SEQ ID NO:21) and EpCAM binding motif RGDPAYQGRFL (SEQ ID NO:34) are simultaneously presented to bind multiple receptors in order to capture metastatic tumor cells from mixed cell population. In another embodiment, cadherin binding motif DQNDN (SEQ ID NO:31) and EpCAM binding motif RGDPAYQGRFL (SEQ ID NO:34) are simultaneously presented to bind multiple receptors in tumor cells.

Remodeling of the ECM and changes to cell interactions with the ECM are essential in the initiation and progression of EMT. Increased α5β1 integrin expression during EMT increases cell adhesion to fibronectin, the expression of which is also activated during EMT and promotes cell migration. The increased expression of α1β1 or α2β1 integrins and their interactions with type I collagen facilitate the disruption of E-cadherin complexes and the nuclear translocation of β-catenin (see, Samy Lamouille, et al., Molecular mechanisms of epithelial-mesenchymal transition, Nat. Rev. Mol. Cell. Biol. 2014 Mar; 15(3):178-196).

Recently, Reticker found that metastatic cells selectively associate with fibronectin when in combination with galectin or laminin and showed that the interaction between fibronectin and galectin/laminin are mediated in part by α3β1integrin (see, Reticker-Flynn et al., A combinatorial extracellular matrix platform identifies cell-extracellular matrix interactions that correlate with metastasis, Nat. Commun. 2012; 3:1122).

Also described is a synthetic tumor microenvironment surface that presents α3β1 or α5β1 integrin binding motif to capture or sort metastatic tumor cells from a mixed cell population. Any suitable α5β1 integrin binding motif can be selected from RGD (SEQ ID NO:15), RGDSGSGSGRGDSGSGSGRGD (SEQ ID NO:16), GRGDSP (SEQ ID NO:36), PHSRN-RGDSP (SEQ ID NO:37), SPPRRARVT (SEQ ID NO:38), WQPPRARI (SEQ ID NO:39), or its combination of two different α5β1 integrin binding motifs. Any suitable α3β1 integrin binding motif can be selected from IKVAV (SEQ ID NO:40), YIGSR (SEQ ID NO:54), PPFLMLLKGSTR (SEQ ID NO:55), SLVRNRRVITTIQ (SEQ ID NO:56).

Increasing evidence from the analysis of isolated CTCs has demonstrated significant heterogeneity of EMT markers supporting the concept of EMT as an important feature of invasive cancer cells. (See, Douglas S. Micalizzi, et al., Cancer metastasis through the prism of epithelial-to-mesenchymal transition in circulating tumor cells, Molecular Oncology 11(2017) 770-780.)

It may be possible to select CTC sub-populations using a combination of antibodies, for example, CD45 antibody combined with antibodies for various tumor markers such as HER2 or estrogen receptor. U.S. Patent (see, U.S. App. No. 2012/0100538) discloses a method of isolation of CTC from samples using antibody cocktails composed of two different antibodies binding two different receptors, respectively. However, the antibody cocktails typically used in such tests are generated using immortalized cell lines that may not recapitulate the continuum of changes occurring in CTC (dynamic environmental changes in CTC) released from patient tumors (see, C. V. Pecot (2011), A novel platform for detection of CK+ and CK− CTCs, Cancer Discovery, 1(7):580-586). Accordingly, there is a need for a method able to effectively detect and target rare invasive sub-population of CTCs present in patient samples. (See, U.S. Patent Application No. US2014/0134646.)

Similarly, CTC sub-populations may be selected using a combination of cell binding motif from epithelial rich or mesenchymal rich extracellular microenvironment.

Also described is a synthetic tumor microenvironment that binds α5β1, α6β1 and/or αvβ5 specifically or simultaneously in order to capture or sort tumor cells. Any suitable substrate protein containing peptide ligand to bind integrin α5β1-, αvβ5-, α6β1, or α9β1 specifically or simultaneously to capture or sort tumor cells. In one embodiment, the microenvironment surface provides a substrate protein presenting α5β1 integrin binding motif derived from fibronectin domain III. Any suitable α5β1 integrin activating- or heparin binding motif can be selected from RGD (SEQ ID NO:15), GRGDSP (SEQ ID NO:36), PHSRN-RGDSP (SEQ ID NO:37), SPPRRARVT (SEQ ID NO:38), WQPPRARI (SEQ ID NO:39), or its combination of two different α5β1 integrin binding motifs.

In another embodiment, the tumor microenvironment surface provides a substrate protein presenting syndecan binding motif KNSFMALYLSKGRLVFALG (SEQ ID NO:61) or α6β1 integrin activating motif derived laminin α1 or laminin α5 LG domain to support self-renewal and pluripotency of a stem cell. Any suitable α6β1 integrin activating motif can be selected from GKNTGDHFVLYM (SEQ ID NO:41), VVSLYNFEQTFML (SEQ ID NO:42), RFDQELRLVSYN (SEQ ID NO:43), RLVSYSGVLFFLK (SEQ ID NO:44), ASKAIQVFLLGG (SEQ ID NO:45), VLVRVERATVFS (SEQ ID NO:46), TVFSVDQDNMLE (SEQ ID NO:47), RLRGPQRVFDLH (SEQ ID NO:48), FDLHQNMGSVN (SEQ ID NO:49), QQNLGSVNVSTG (SEQ ID NO:50), SRATAQKVSRRS (SEQ ID NO:51), TWYKIAFQRNRK (SEQ ID NO:52), NRWHSIYITRFG (SEQ ID NO:53), RIQNLLKITNLRIKFVK (SEQ ID NO:62), RKRLQVQLSIRT (SEQ ID NO:63).

A strategy to enrich viable CTCs is to capture a specific portion of CTCs based on their function. A method, which is based on tumor cells' ability to attach and ingest collagen adhesion matrix, has been described as collagen adhesion matrix (CAM) assay (see, J. Lu, T. Fan, Q. Zhao, W. Zeng, E. Zaslaysky, et al. (2010), Isolation of circulating epithelial and tumor progenitor cells with an invasive phenotype from breast cancer patients, Int. J. Cancer 126:669-68). To enrich tumor cells, blood samples were simply transferred into a CAM-coated tube and incubated for several hours. Unattached cells were then washed off and adherent cells were collected (see, P. L. Paris et al. (2009), Functional phenotyping and genotyping of circulating tumor cells from patients with castration resistant prostate cancer, Cancer Lett. 277:164-173). Similarly, Wang, et al. developed an invasion assay to detect CTCs which could invade into Matrigel (see, H. Wang et al. (2015), Detection and enumeration of circulating tumor cells based on their invasive property, Oncotarget 6:27304-27311). These methods do well in preserving the viability of CTCs, thus enabling further expansion of CTCs. However, not all cancer cells in the circulatory system are viable, so this method is specifically developed to capture viable CTCs. The non-viable CTCs, which may still be valuable for diagnosis/prognosis purpose and cancer molecular analysis, will be excluded (see, Tianyu Guo, et al. (2016), Culture of Circulating Tumor Cells-Holy Grail and Big Challenge, Int. J. Cancer Clin. Res. 3:065).

Also described is a surface to enrich leukocytes from biological samples for efficient CTC enrichment. CTCs are outnumbered by leukocytes in the blood, and leukocyte depletion prior to CTC enrichment is less expensive and more efficient as it gives more than 90% leukodepletion of blood along with minimal cell loss. Integrins expressed predominantly by leukocytes consist of an α4 subunit such as α4β1 and α4β7, and β2 subunit coupled with one of several a subunit counterpart (see, Young-Min Hyun, et al., Immunol. Res. 2009 Volume 45, Issue 2-3, p195-208). A surface presenting α4 subunit β2 subunit binding peptide may be used for depletion of leukocyte prior to CTC enrichment.

Any suitable α4β1 integrin activating motif can be selected from EILDVPST (SEQ ID NO:57), LDVPS (SEQ ID NO:59), EDGIHEL (SEQ ID NO:60).

The disclosure further provides a synthetic tumor microenvironment for in situ expansion of captured metastatic cells such as circulating tumor cells from patient sample. According to the disclosure, a synthetic tumor microenvironment surface does not require the use of growth factor supplements for captured CTC expansion. The synthetic tumor microenvironment itself provides favorable environment for CTC expansion and/or CTC cluster formation within in two or three days with an overall cluster formation success rate of ˜80%.

As used herein, “tumor microenvironment array” refers to a combination of two or more microlocations. Preferably, an array is comprised of microlocations in addressable rows and columns. The layout of microenvironment arrays produced according to the invention can vary, dependent upon the metastatic stage of tumor cells.

The invention provides for a device of tumor microenvironment array comprising:
(a) preparing composition comprising one or more cell binding motif;
(b) placing a the composition on surface of a substrate to form Cell

Capturing Island; and
(c) obtaining the synthetic tumor microenvironment surface array.

In one embodiment of this invention, a synthetic tumor microenvironment array is provided. The array is a 96-well microwell plate consisting of 8×12-well. Each well within a strip is coated with a different cell binding ligand containing composition to generate different tumor microenvironment. Tumor cells of interest can be seeded onto each well, whereby tumor cells are captured or sorted on different tumor microenvironment surface. A synthetic tumor microenvironment that induces a desirable cellular behavior or mechanism such as EMT, invasion, metastasis can be identified and designed from the assay utilizing this tumor microenvironment array.

The tumor microenvironment array can be used in high throughput screening (HTS) to identify combinatorial cell binding motif to engineer optimal synthetic tumor microenvironment that can specifically, selectively, simultaneously or sequentially generate signaling pathway to mediate invasion, intravasation, survival of metastatic tumor cells.

The following examples are provided to demonstrate preferred embodiments of the disclosure and the invention is not intended to be limited in scope by the specific embodiments described herein, which are intended for the purposes of exemplification only. Functionally equivalent products, compositions and methods are clearly within the scope of the invention, as described herein.

### EXAMPLES

### Example 1. Synthetic Tumor Microenvironment Surface

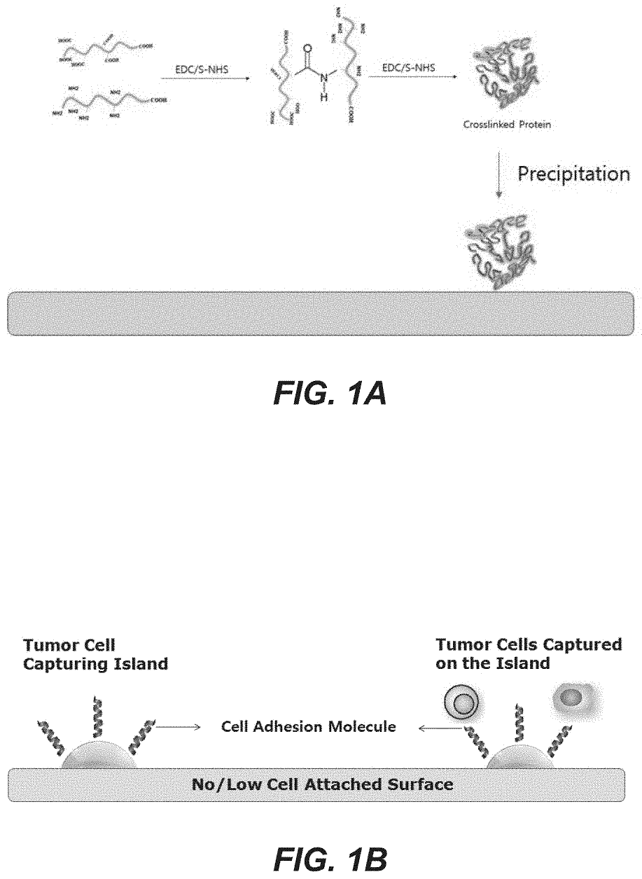

Synthetic tumor microenvironment surfaces were prepared by immobilizing cell binding peptide, single or in combination, on neutral or hydrophobic surface. A general scheme of the tumor microenvironment surface functionalized with cell adhesion peptide motifs is shown in FIGS. 1A and 1B. The cell binding peptide may comprise integrin binding motif, cadherin binding motif, EpCAM binding motif in order to induce binding of tumor cells at different metastatic stage on the synthetic tumor microenvironment surface.

MAPTrix™ based particles, Cell Capturing Island, were formed by reaction of the carboxyl group of MAPTrix™ activated with 1-ethyl-3-(3-dimethylaminopropyl) carbodiimides/sulfo-N-hydroxysulfosuccinimide (EDC/S-NHS) on the C-terminus with the amino groups of the MAPTrix™.

1-Ethyl-3-[3-Dimethylaminopropyl]carbodiimide hydrochloride (EDC) solution is prepared by dissolving 10 mg of EDC in 1 ml of sodium bicarbonate buffer (10 mM, pH 6.5). 5 mg of solid sulfo-N-hydroxysulfosuccinimide (S-NHS) is added to the EDC solution. The EDC/S-NHS solution is added to the MAPTrix™ solution to activate carboxyl group on the MAPTrix™ for 30 minutes. 0.1 mg of MAPTrix™ having RGD (SEQ ID NO:15), GRGDSP (SEQ ID NO:36), GFPGER (SEQ ID NO:21), DQNDN (SEQ ID NO:31), LFSHAVSSNG (SEQ ID NO:30), ADTPPV (SEQ ID NO:32), RGDPAYQGRFL (SEQ ID NO:34) motif dissolved in 1 mL distilled water was added to 24-well plate. Crosslinking is carried out at ambient temperature for 30 minutes to get crosslinked MAPTrix™ particle presenting cell binding motif. The particles were precipitated on the surface to form Cell Capturing Island.

As seen in the FIG. 2, the MAPTrix™ particles were observed. Depending on the concentration of MAPTrix™, the particle size ranged from 0.5 to 5 μm.

### Example 2. Capturing of in Vivo Like Circulating Tumor Cell

MCF-7 and MD-MBA-231 cells were purchased from ATCC (Manassas, Va.). MCF-7 cells were cultured on the synthetic tumor microenvironment surface as prepared in EXAMPLE 1 in 100% horse serum (v/v) to mimic in vivo like CTC environment and 1% (v/v) penicillin/streptomycin in a humidified incubator at 37° C. and 5% CO₂. MD-MBA-231 cells were cultured on the synthetic tumor microenvironment surface as prepared in EXAMPLE 1 in 100% horse serum and 1% (v/v) penicillin/streptomycin in a humidified incubator at 37° C. and 5% CO₂.

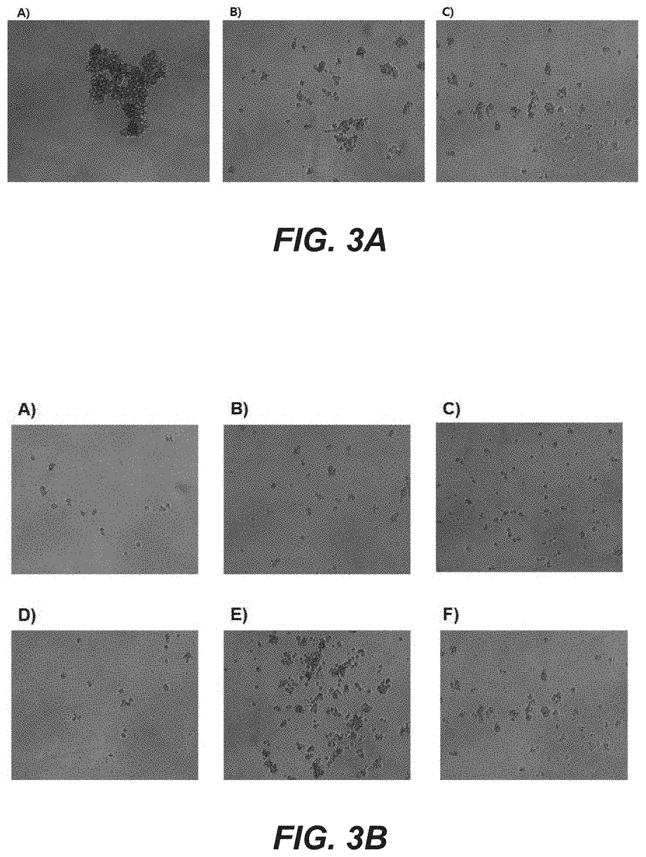

After 6 hours incubation, the medium was exchanged with fresh RPMI media and unattached cells were removed during the media exchange. Optical microscope observation revealed substrate-attached cells and unattached cells floating in the culture medium as seen in FIGS. 3A and 3B. Three different integrin binding peptide motifs and one cadherin binding motif were identified to capture MDA-MB-213, a CTC-like metastatic cancer cell. Collagen derived peptide motif GFGPER (SEQ ID NO:21) in FIG. 3B (A) and cadherin derived peptide motif DQNDN (SEQ ID NO:31) in FIG. 3B (B) and ADTPPV (SEQ ID NO:32) in FIG. 3B (C), and RGD motif (SEQ ID NO:15) in FIG. 3B (D) supported strong adhesion to MDA-MB-231 while other motifs such as fibronectin derived peptide motif GRGDSP (SEQ ID NO:36) in FIG. 3B (E) did not adhere to MDA-MB-231.

### Example 3. Construction of Synthetic Tumor Microenvironment Array

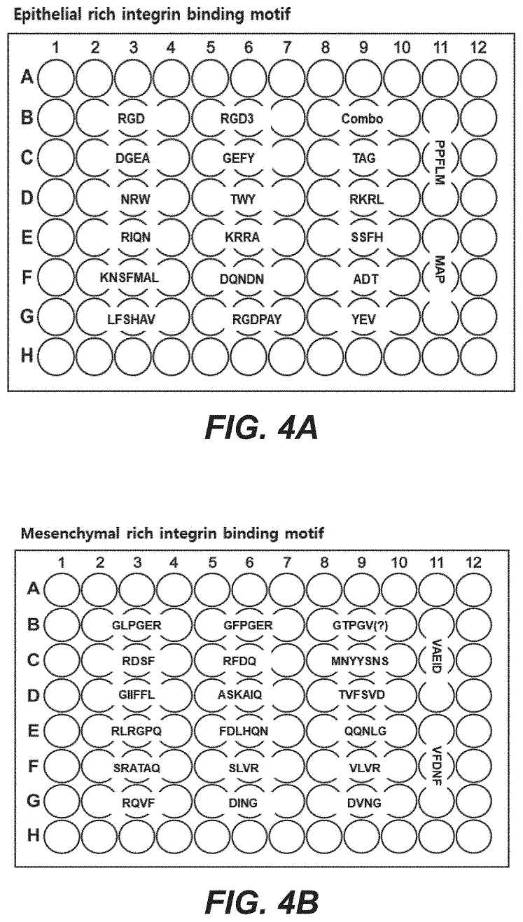

To create a highly sensitive surface by utilizing the multivalent effect, the surface presenting combination of different cell adhesion motif is required as illustrated in FIGS. 4A and 4B. Mixtures of integrin binding peptide and E-cadherin, integrin binding peptide and EpCAM were immobilized at various ratios under the same condition described above. A fixed concentration of integrin binding peptide at 0.1 mg/mL was used with various amounts of E-cadherin peptide and EpCAM binding peptide. The final total weights (in mg) of integrin and E-cadherin/EpCAM binding peptide were 1.5:0, 1.5:0.3, 1.5:1.5, and 1.5:7.5.

### Example 4. Design of Tumor Microenvironment Array of Combinatorial Presentation of Different Cell Binding Ligand

Several arrays of 24 different tumor microenvironment were prepared as represented in FIGS. 4A and 4B. A representative array surface to present collagen, fibronectin and laminin derived peptide motif to bind integrin, E-cadherin, and EpCAM binding motif was screened to identify tumor microenvironmental surface that promote capturing tumor cells.

For tumor microenvironment array, stock solutions of each ECM mimetic were suspended and dissolved sodium bicarbonate buffer (0.1 M, pH 6.5) at 0.1 mg/mL. ECM mimetic solutions were then used in single or mixed in 24 different combinations in a 96-microwell plate. The layout for epithelial and mesenchymal microenvironment array was represented in FIGS. 4A and 4B, respectively.

### Example 5. Cell Adhesion Assay of CTC Like Cells and Leukocytes on the Tumor Microenvironment Surface

To identify cell binding ligand for non-metastatic and metastatic CTC, MCF-7, MDA-MB-231 and HL60 cells were cultured on epithelial surface and mesenchymal surface in accordance with the same culture conditions and capturing procedure in EXAMPLE 4.

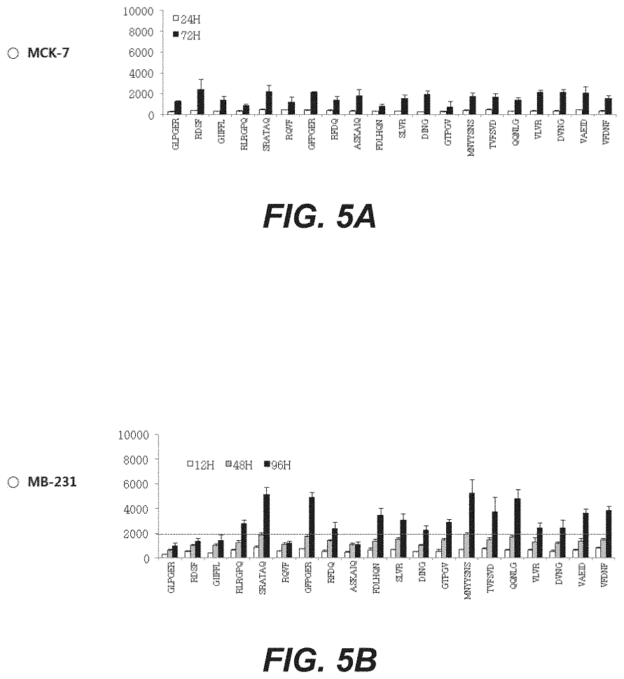

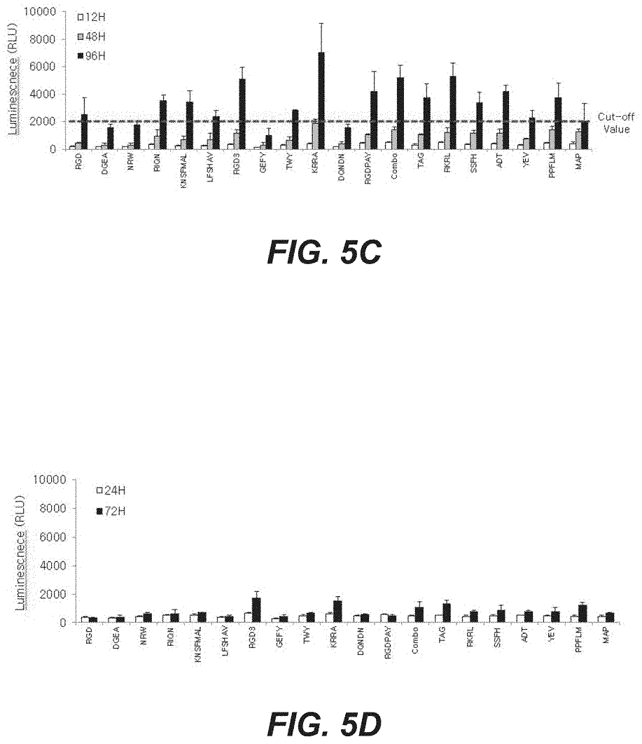

FIGS. 5A-5D presented the cell adhesion profiling of two MCF-7 and MDA-MB-231 on epithelial surface and mesenchymal surface. As seen in FIGS. 5A and 5C, MCF-7 showed strong adhesion on epithelial surface (FIG. 5C) and poor adhesion on mesenchymal surface (FIG. 5A). Some cell binding ligands RGD-SGSGSG-RGD-SGSGSG-RGD (SEQ ID NO:16), RKRLQVQLSIRT (SEQ ID NO:63), KNSFMALYLSKGRLVFALG (SEQ ID NO:61). MDA-MB-231 showed strong adhesion on mesenchymal surface (FIG. 5B) and poor adhesion on epithelial surface (FIG. 5D). Some cell binding ligands GFPGER (SEQ ID NO:21), SRATAQKVSRRS (SEQ ID NO:51) MMYYSNS (SEQ ID NO:17), TVFSDQDNMLE (SEQ ID NO:47) and QQNLGSVNVSTG (SEQ ID NO:50), RIQNLLKITNLRIKFVK (SEQ ID NO:62) were identified for capturing metastatic cancer cells.

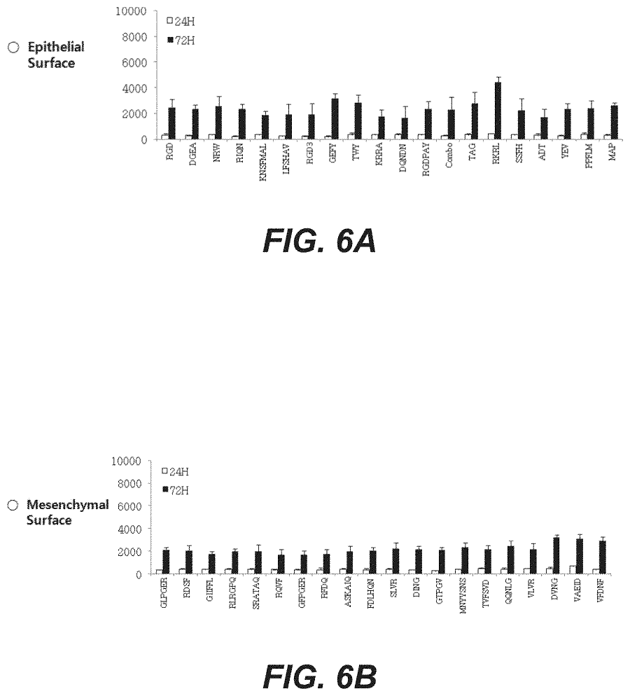

However, leukocytes showed poor adhesion on both epithelial and mesenchymal surface as shown in FIGS. 6A and 6B.

### Example 6. Synergistic Effect of Combinatorial Presentation of Different Cell Binding Ligands on CTC Capturing

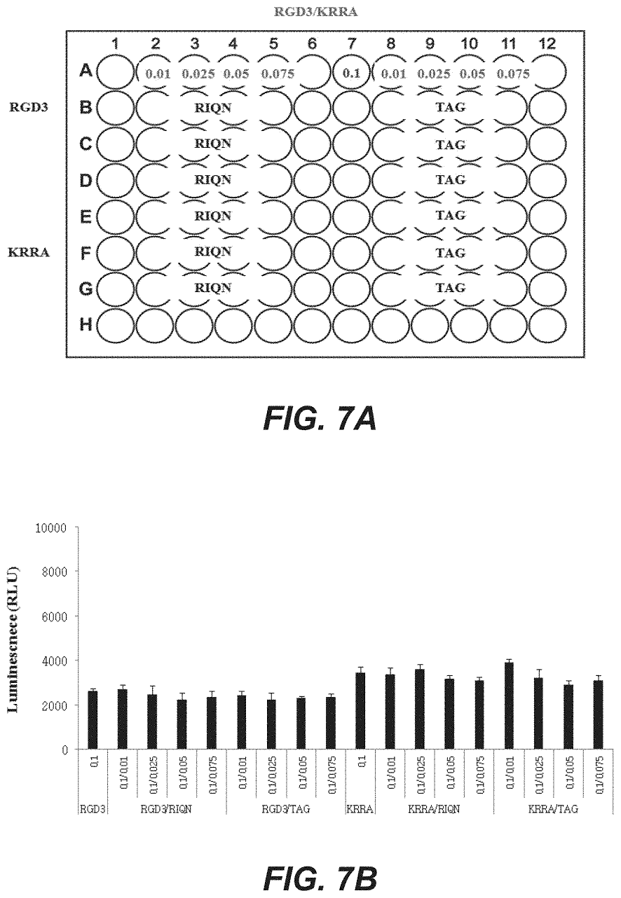

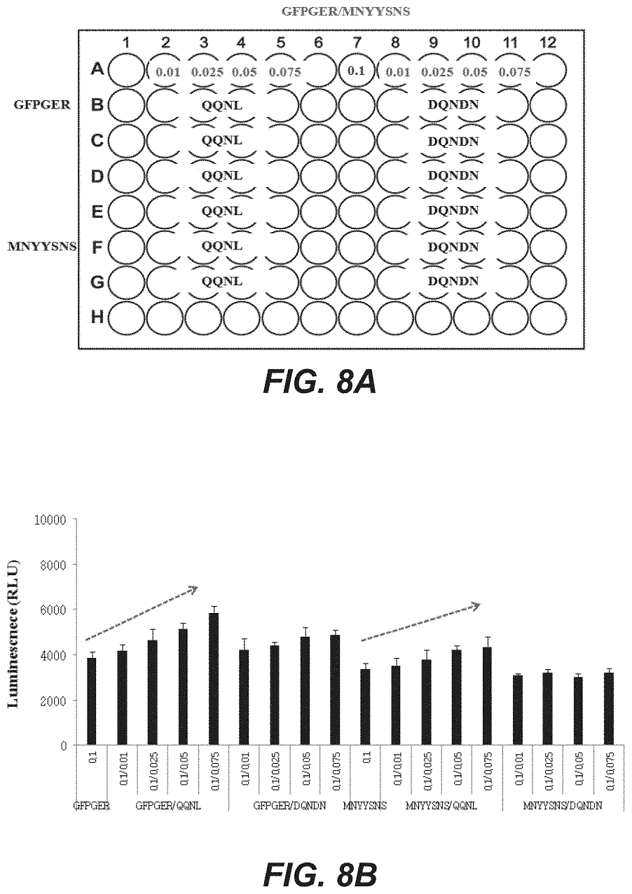

Cell binding ligands with strong adhesion to metastatic and non-metastatic cancer cells were selected to design combinatorial presentation as seen in FIG. 7A (epithelial surface) and FIG. 8A (mesenchymal surface). Binding of two different cell binding ligands to cancer cells would enhance the capturing efficiency.

To identify synergic effect of two cell binding ligands for non-metastatic and metastatic CTCs, MCF-7 and MDA-MB-231 cells were cultured on epithelial surface (FIG. 7A) and mesenchymal surface (FIG. 8A) in accordance with the same culture conditions and capturing procedure in EXAMPLE 4.

No synergistic effect was observed in epithelial surface, but significant synergistic effect was observed in mesenchymal surface as seen in FIG. 8B.

### Example 7. In Situ Expansion of CTC-Like Cells After Capturing

Due to the low frequency in the blood, heterogeneity and poor survival in general culture condition of circulating tumor cells, ex vivo expansion of CTC is required to characterize in transcriptomic, genomic and functional terms for clinical application.

Metastatic and non-metastatic cancer cells, MDA-MB-231 and MCF-7, respectively, were cultured on collagen-cadherin binding peptide motif coated surface or collagen-EpCam binding peptide motif coated surface in accordance with the same culture conditions and capturing procedure in EXAMPLE 4.

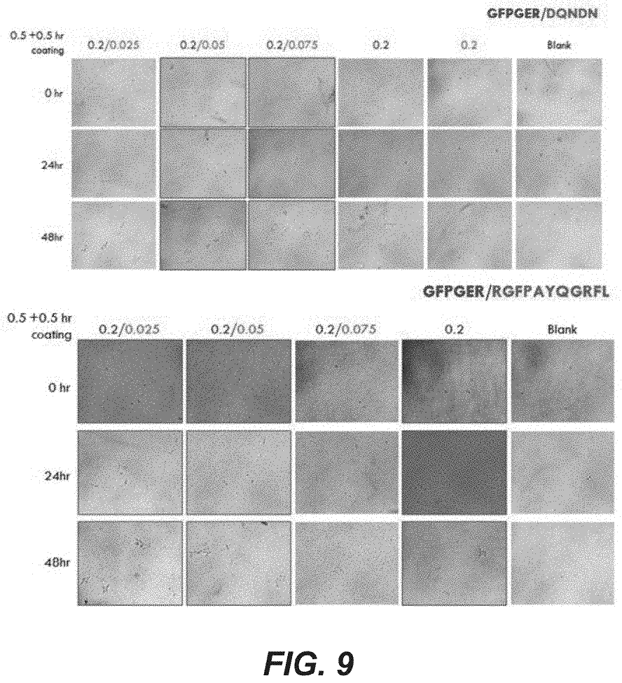

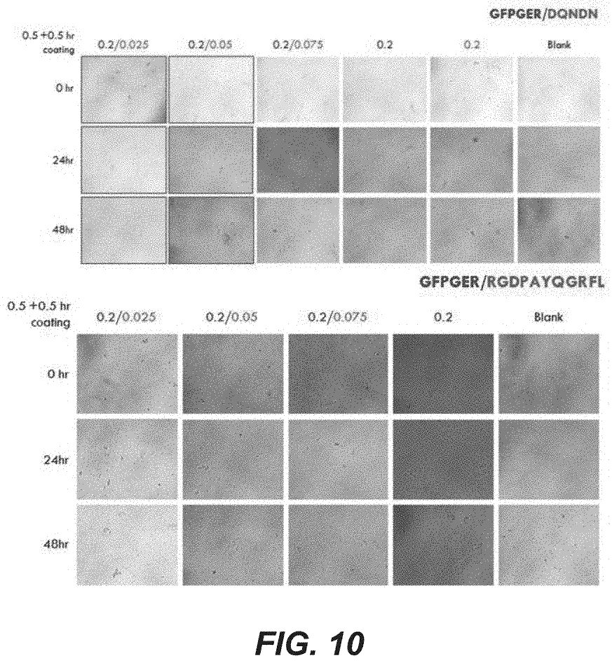

After 96 hours incubation with daily exchange of media, growing cells were observed by optical microscope. FIGS. 9 and 10 showed the effect of combinatorial cell binding ligand motifs on the expansion of captured MDA-MB-231 and MCF-7 on the tumor microenvironment surface.

FIG. 9 showed metastatic cancer cells (MDA-MB-231) were expanded on both collagen-cadherin and collagen-EpCAM surface but MCF-7 cells were expanded only on collagen-cadherin surface as seen in FIG. 10.

Significant synergy was observed in collagen-cadherin surface where two peptide motifs GFPGER (SEQ ID NO:21) and DQNDN (SEQ ID NO:31) were presented.

### Example 8. Capturing Leukocytes From the Synthetic Tumor Microenvironment

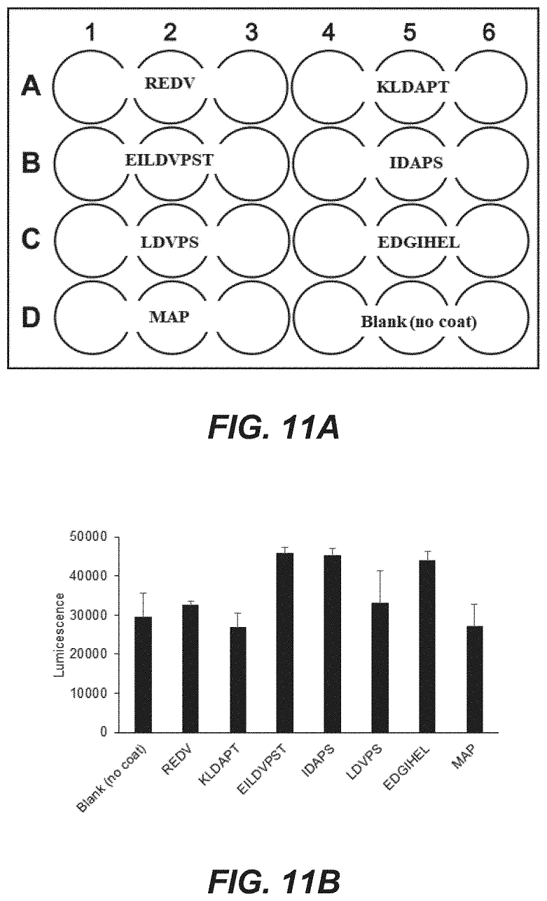

For a leukocyte capturing array, stock solutions of each ECM mimetic were suspended and dissolved sodium bicarbonate buffer (0.1 M, pH 6.5) at 0.1 mg/mL. ECM mimetic solutions were then dispensed in single or mixed in XX different combinations in a 24-microwell plate. The layout for capturing of leukocytes microenvironment array was represented in FIG. 11A.

HL-60 cells, peripheral blood cell-derived leukocyte cells, were purchased from ATCC (Manassas, Va.). HL-60 cells were cultured in RPMI media supplemented with 10% (v/v) fetal bovine serum (FBS) and 1% (v/v) penicillin/streptomycin in a humidified incubator at 37° C. and 5% CO2. To test leukocyte capturing, HL-60 cells were cultured in 100% (v/v) horse serum and 1% (v/v) penicillin/streptomycin with ECM mimetic peptide coated plates. After 6 hours incubation, cultured media was removed and fresh RPMI media with 10% FBS was added. Cell viability was determined by CellTiter Glo Luminescence assay (Promega, Madison, Wis.). Among tested ECM mimetic peptides, EILDVPST, IDAPS and EDGIHEL motif showed leukocyte capturing (FIG. 11B).

These results suggest synthetic tumor microenvironment could capture leukocytes in in vivo mimic condition.

### Example 9. Effect of Synthetic Tumor Microenvironment on the Cancer Cell Stemness Potential Expanded on the Synthetic Tumor Microenvironment Surface

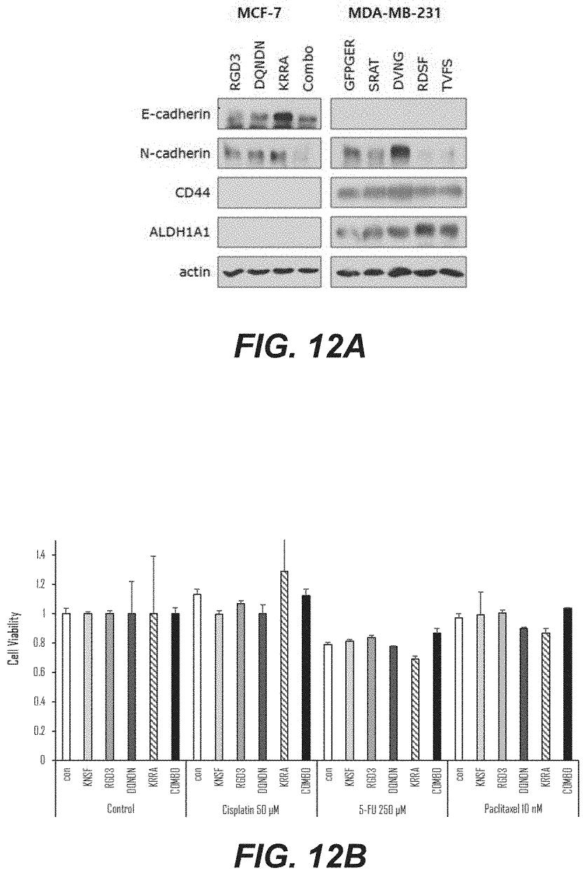

The stemness of synthetic ECM mimetic motif-captured cancer cells was determined by two different methods, western blot and CellTiter Glo-luminescence assay. The layout, used for cancer cell stemness testing, was represented in FIGS. 12A and 12B. Following previous in vitro capturing cancer method, cancer cells, MDA-MB-231, were captured by ECM mimetic peptides and expression level of stemness markers, CD44 and ALDH1A1 was determined by Western blot with specific antibodies (Abcam, Cambridge, UK). Cancer stemness markers were detected in five different EMC mimetic peptides-captured cancer cells (FIG. 12A). Cancer drug resistance is the characteristics of cancer stem cells. Based on synthetic microenvironment, cancer drug resistance was monitored using cisplatin and 5-FU (Sigma-Aldrich, St. Louis, Mo.). MDA-MB-231 was captured by synthetic microenvironment and treated with cisplatin and 5-FU for 48 hour. Compared with conventional cultured and control peptide-captured cells, two different EMC mimetic peptides induced drug resistance (FIG. 12B). Drug sensitivity was determined by CellTiter Glo-luminescence assay. Cells captured/expanded on epithelial surface have no CSC marker while cells on mesenchymal surface showed high CSC marker (CD44 & ALDH1A1) expression. These results suggest synthetic tumor microenvironment could induce and maintain the stemness of cancer stem cells.

1. A surface coated with particles presenting tumor microenvironment surface, wherein cells can bind only to the particles and cells unbound to the particles are forced to be suspended.

2. The surface of claim 1, wherein the tumor microenvironment presents at least one or more cell binding ligands that bind specifically to at least one or more cell adhesion molecules highly expressed in cancer cells of interest.

3. The surface of claim 1, wherein the cell adhesion molecules are selected from integrins, cadherins or EpCAM.

4. The surface of claim 2, wherein the cell binding ligands are selected from integrin binding peptide, cadherin binding peptide or EpCAM binding peptide.

5. The surface of claim 1, wherein the surface is an electrically neutral or hydrophobic surface.

6. The surface of claim 5, wherein the surface is a low cell attachment surface.

7. The surface of claim 3, wherein the integrins are selected from αvβ6, α2β1, α3β1, α5β1, or α5β1.

8. The surface of claim 7, wherein the integrin binding peptide for αvβ6 is selected from RGD (SEQ ID NO:15), RGD-SGSGSG-RGD-SGSGSG-RGD (SEQ ID NO:16), or MNYYSNS (SEQ ID NO:17).

9. The surface of claim 7, wherein the integrin binding peptide for α2β1 is selected from GLSGER (SEQ ID NO:18), GASGER (SEQ ID NO:19), GQRGER (SEQ ID NO:20), GFPGER (SEQ ID NO:21), GLPGER (SEQ ID NO:22), DGEA (SEQ ID NO:23), GPAGKDGEAGAQG (SEQ ID NO:24), TAGSCLRKFSTM (SEQ ID NO:25), MFKKPTPSTLKAGELR (SEQ ID NO:26), LAGSCLARFSTM (SEQ ID NO:27) or GEFYFDLRLKGDK (SEQ ID NO:28).

10. The surface of claim 7, wherein the integrin binding peptide for α5β1 is selected from RGD (SEQ ID NO:15), RGDSGSGSGRGDSGSGSGRGD (SEQ ID NO:16), GRGDSP (SEQ ID NO:36), PHSRN-RGDSP (SEQ ID NO:37), SPPRRARVT (SEQ ID NO:38), WQPPRARI (SEQ ID NO:39).

11. The surface of claim 7, wherein the integrin binding peptide for α3β1 is selected from IKVAV (SEQ ID NO:40), YIGSR (SEQ ID NO:54), PPFLMLLKGSTR (SEQ ID NO:55) or SLVRNRRVITTIQ (SEQ ID NO:56).

12. The surface of claim 7, wherein the integrin binding peptide for α6β1 is selected from the group consisting of GKNTGDHFVLYM (SEQ ID NO:41), VVSLYNFEQTFML (SEQ ID NO:42), RFDQELRLVSYN (SEQ ID NO:43), RLVSYSGVLFFLK (SEQ ID NO:44), ASKAIQVFLLGG (SEQ ID NO:45), VLVRVERATVFS (SEQ ID NO:46), TVFSVDQDNMLE (SEQ ID NO:47), RLRGPQRVFDLH (SEQ ID NO:48), FDLHQNMGSVN (SEQ ID NO:49), QQNLGSVNVSTG (SEQ ID NO:50), SRATAQKVSRRS (SEQ ID NO:51), TWYKIAFQRNRK (SEQ ID NO:52), NRWHSIYITRFG (SEQ ID NO:53), RIQNLLKITNLRIKFVK (SEQ ID NO:62), and RKRLQVQLSIRT (SEQ ID NO:63).

13. The surface of claim 7, wherein the cadherin binding peptide is selected from the group consisting of SHAVSS (SEQ ID NO:29), LFSHAVSSNG (SEQ ID NO:30), DQNDN (SEQ ID NO:31), ADTPPV (SEQ ID NO:32), QGADTPPVGV (SEQ ID NO:33), LRAHAVDVNG (SEQ ID NO:64), and a combination of two or more E-cadherin binding motifs.

14. The surface of claim 7, wherein the EpCAM binding peptide is selected from the group consisting of RGDPAYQGRFL (SEQ ID NO:34), YEVHTYYLD (SEQ ID NO:35), and a combination thereof.

15. The surface of claim 2, wherein the cell adhesion ligands are composed of two different ligands.

16. The surface of claim 15, wherein the two different ligands are integrin α2β1 binding peptide and cadherin binding peptide.

17. The surface of claim of 16, wherein the integrin α2β1 binding peptide is selected from GLSGER (SEQ ID NO:18), GASGER (SEQ ID NO:19), GQRGER (SEQ ID NO:20), GFPGER (SEQ ID NO:21), GLPGER (SEQ ID NO:22), DGEA (SEQ ID NO:23), GPAGKDGEAGAQG (SEQ ID NO:24), TAGSCLRKFSTM (SEQ ID NO:25), MFKKPTPSTLKAGELR (SEQ ID NO:26), LAGSCLARFSTM (SEQ ID NO:27) or GEFYFDLRLKGDK (SEQ ID NO:28) and the cadherin binding peptide is selected from SHAVSS (SEQ ID NO:29), LFSHAVSSNG (SEQ ID NO:30), DQNDN (SEQ ID NO:31) or ADTPPV (SEQ ID NO:32), QGADTPPVGV (SEQ ID NO:33).

18. The surface of claim 15, wherein the α2β1 binding peptide is GFPGER (SEQ ID NO:21) and the cadherin binding peptide is DQNDN (SEQ ID NO:31).

19. A surface coated with particles presenting leukocyte integrin binding motif, wherein leukocytes can bind only to the particles and other cells unbound to the particles are forced to be suspended.


SEQUENCE LISTING (64 sequences)

SEQ ID NO: 1 - protein, 75 aa, Mytilus edulis
SSEEYKGGYYPGNAYHYHSGGSYHGSGYHGGYKGKYYGKAKKYYYKYKNSGKYKYLKKARKYHRKGYKKYYGGSS

SEQ ID NO: 2 - protein, 75 aa, Mytilus edulis; identical to SEQ ID NO: 1

SEQ ID NO: 3 - protein, 71 aa, Mytilus coruscus
YDDYSDGYYPGSAYNYPSGSHWHGHGYKGKYYGKGKKYYYKFKRTGKYKYLKKARKYHRKGYKKHYGGSSS

SEQ ID NO: 4 - protein, 76 aa, Mytilus galloprovincialis
SSEEYKGGYYPGNTYHYHSGGSYHGSGYHGGYKGKYYGKAKKYYYKYKNSGKYKYLKKARKYHRKGYKKYYGGGSS

SEQ ID NO: 5 - protein, 56 aa, Artificial Sequence
  Barnacle-derived adhesive protein
MRVILFAMLIGGSLACQNRLETLVQEATGNAGDLSTNVHEECNSQVGTFNAVHAPQ

SEQ ID NO: 6 - protein, 10 aa, Artificial Sequence
  Model peptide of the tandem repeat decapeptide derived from foot protein 1 (FP-1, Mytilus edulis)
AKPSYPPTYK

SEQ ID NO: 7 - protein, 20 aa, Artificial Sequence
  2 times repeated sequence derived from foot protein 1 (FP-1, Mytilus edulis)
AKPSYPPTYKAKPSYPPTYK

SEQ ID NO: 8 - protein, 60 aa, Artificial Sequence
  6 times repeated sequence derived from foot protein 1 (FP-1, Mytilus edulis)
AKPSYPPTYKAKPSYPPTYKAKPSYPPTYKAKPSYPPTYKAKPSYPPTYKAKPSYPPTYK

SEQ ID NO: 9 - protein, 39 aa, Mytilus californianus
EVHACKPNPCKNNGRCYPDGKTGYKCKCVGGYSGPTCAC

SEQ ID NO: 10 - protein, 52 aa, Mytilus edulis
ADYYGPKYGPPRRYGGGNYNRYGGSRRYGGYKGWNNGWKRGRWGRKYYEFEF

SEQ ID NO: 11 - protein, 46 aa, Mytilus galloprovincialis
ADYYGPKYGPPRRYGGGNYNRYGRRYGGYKGWNNGWKRGRWGRKYY

SEQ ID NO: 12 - protein, 60 aa, Mytilus californianus
GHVHRHRVLHKHVHNHRVLHKHLHKHQVLHGHVHRHQVLHKHVHNHRVLHKHLHKHQVLH

SEQ ID NO: 13 - protein, 194 aa, Artificial Sequence
  Hybrid mussel adhesive protein (FP-151, MEFP-5 based Kollodis)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 14 - protein, 196 aa, Artificial Sequence
  Hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 15 - protein, 3 aa, Artificial Sequence
  Fibronectin-derived peptide
RGD

SEQ ID NO: 16 - protein, 21 aa, Artificial Sequence
  FIbronectin-derived peptide
RGDSGSGSGRGDSGSGSGRGD

SEQ ID NO: 17 - protein, 7 aa, Artificial Sequence
  Collagen-derived peptide
MNYYSNS

SEQ ID NO: 18 - protein, 6 aa, Artificial Sequence
  Collagen-derived peptide
GLSGER

SEQ ID NO: 19 - protein, 6 aa, Artificial Sequence
  Collagen-derived peptide
GASGER

SEQ ID NO: 20 - protein, 6 aa, Artificial Sequence
  Collagen-derived peptide
GQRGER

SEQ ID NO: 21 - protein, 6 aa, Artificial Sequence
  Collagen-derived peptide
GFPGER

SEQ ID NO: 22 - protein, 6 aa, Artificial Sequence
  Collagen-derived peptide
GLPGER

SEQ ID NO: 23 - protein, 4 aa, Artificial Sequence
  Collagen-derived peptide
DGEA

SEQ ID NO: 24 - protein, 13 aa, Artificial Sequence
  Collagen-derived peptide
GPAGKDGEAGAQG

SEQ ID NO: 25 - protein, 12 aa, Artificial Sequence
  Collagen-derived peptide
TAGSCLRKFSTM

SEQ ID NO: 26 - protein, 16 aa, Artificial Sequence
  Collagen-derived peptide
MFKKPTPSTLKAGELR

SEQ ID NO: 27 - protein, 12 aa, Artificial Sequence
  Collagen-derived peptide
LAGSCLARFSTM

SEQ ID NO: 28 - protein, 12 aa, Artificial Sequence
  Collagen-derived peptide
LAGSCLARFSTM

SEQ ID NO: 29 - protein, 6 aa, Artificial Sequence
  E-cadherin-derived peptide
SHAVSS

SEQ ID NO: 30 - protein, 10 aa, Artificial Sequence
  E-cadherin-derived peptide
LFSHAVSSNG

SEQ ID NO: 31 - protein, 5 aa, Artificial Sequence
  E-cadherin-derived peptide
DQNDN

SEQ ID NO: 32 - protein, 6 aa, Artificial Sequence
  E-cadherin-derived peptide
ADTPPV

SEQ ID NO: 33 - protein, 10 aa, Artificial Sequence
  E-cadherin-derived peptide
QGADTPPVGV

SEQ ID NO: 34 - protein, 11 aa, Artificial Sequence
  Synthetic EpCAM binding peptide
RGDPAYQGRFL

SEQ ID NO: 35 - protein, 9 aa, Artificial Sequence
  Synthetic EpCAM binding peptide
YEVHTYYLD

SEQ ID NO: 36 - protein, 6 aa, Artificial Sequence
  Fibronectin-derived peptide
GRGDSP

SEQ ID NO: 37 - protein, 10 aa, Artificial Sequence
  Fibronectin-derived peptide
PHSRNRGDSP

SEQ ID NO: 38 - protein, 9 aa, Artificial Sequence
  Fibronectin-derived peptide
SPPRRARVT

SEQ ID NO: 39 - protein, 8 aa, Artificial Sequence
  Fibronectin-derived peptide
WQPPRARI

SEQ ID NO: 40 - protein, 5 aa, Artificial Sequence
  Laminin-derived peptide
IKVAV

SEQ ID NO: 41 - protein, 12 aa, Artificial Sequence
  Laminin-derived peptide
GKNTGDHFVLYM

SEQ ID NO: 42 - protein, 13 aa, Artificial Sequence
  Laminin-derived peptide
VVSLYNFEQTFML

SEQ ID NO: 43 - protein, 12 aa, Artificial Sequence
  Laminin-derived peptide
RFDQELRLVSYN

SEQ ID NO: 44 - protein, 13 aa, Artificial Sequence
  Laminin-derived peptide
RLVSYSGVLFFLK

SEQ ID NO: 45 - protein, 12 aa, Artificial Sequence
  Laminin-derived peptide
ASKAIQVFLLGG

SEQ ID NO: 46 - protein, 12 aa, Artificial Sequence
  Laminin-derived peptide
VLVRVERATVFS

SEQ ID NO: 47 - protein, 12 aa, Artificial Sequence
  Laminin-derived peptide
TVFSVDQDNMLE

SEQ ID NO: 48 - protein, 12 aa, Artificial Sequence
  Laminin-derived peptide
RLRGPQRVFDLH

SEQ ID NO: 49 - protein, 11 aa, Artificial Sequence
  Laminin-derived peptide
FDLHQNMGSVN

SEQ ID NO: 50 - protein, 12 aa, Artificial Sequence
  Laminin-derived peptide
QQNLGSVNVSTG

SEQ ID NO: 51 - protein, 12 aa, Artificial Sequence
  Laminin-derived
SRATAQKVSRRS

SEQ ID NO: 52 - protein, 12 aa, Artificial Sequence
  Laminin-derived peptide
TWYKIAFQRNRK

SEQ ID NO: 53 - protein, 12 aa, Artificial Sequence
  Laminin-derived peptide
NRWHSIYITRFG

SEQ ID NO: 54 - protein, 5 aa, Artificial Sequence
  Laminin-derived peptide
YIGSR

SEQ ID NO: 55 - protein, 12 aa, Artificial Sequence
  Laminin-derived peptide
PPFLMLLKGSTR

SEQ ID NO: 56 - protein, 13 aa, Artificial Sequence
  Laminin-derived peptide
SLVRNRRVITTIQ

SEQ ID NO: 57 - protein, 8 aa, Artificial Sequence
  Fibronectin-derived peptide
EILDVPST

SEQ ID NO: 58 - protein, 5 aa, Artificial Sequence
  Fibronectin-derived peptide
IDAPS

SEQ ID NO: 59 - protein, 5 aa, Artificial Sequence
  Fibronectin-derived peptide
LDVPS

SEQ ID NO: 60 - protein, 7 aa, Artificial Sequence
  Fibronectin-derived peptide
EDGIHEL

SEQ ID NO: 61 - protein, 19 aa, Artificial Sequence
  Laminin-derived peptide
KNSFMALYLSKGRLVFALG

SEQ ID NO: 62 - protein, 17 aa, Artificial Sequence
  Laminin-derived peptide
RIQNLLKITNLRIKFVK

SEQ ID NO: 63 - protein, 17 aa, Artificial Sequence
  Laminin-derived peptide
RIQNLLKITNLRIKFVK

SEQ ID NO: 64 - protein, 10 aa, Artificial Sequence
  E-cadherin-derived peptide
LRAHAVDVNG